(19) United States
(12) Patent Application Publication
(10) Pub. No.: US 2020/0056988 A1
(43) Pub. Date: Feb. 20, 2020

(54) METHOD OF DETERMINING THE EFFICACY OF ANTIMICROBIALS

(71) Applicant: Wisconsin Alumni Research Foundation, Madison, WI (US)

(72) Inventors: Daniel Elmer Butz, Madison, WI (US); Ann P. O'Rourke, Madison, WI (US); Mark E. Cook, Madison, WI (US)

(21) Appl. No.: 16/540,627

(22) Filed: Aug. 14, 2019

Related U.S. Application Data
(60) Provisional application No. 62/764,874, filed on Aug. 16, 2018.

(51) Int. Cl. G01N 21/3504; G01N 33/497

(52) U.S. Cl. CPC G01N 21/3504; G01N 33/497

(57) ABSTRACT

A method of determining efficacy of an antimicrobial treatment in a subject includes calculating a breath delta value (BDV) for each of at least six breath samples acquired from the subject over a 24 hour period starting from when the subject has been administered the antimicrobial treatment, calculating a mean standard deviation of BDV (SD BDV) across the six or more breath samples; and determining that the antimicrobial treatment is effective when the SD BDV is less than or equal to 0.46, or determining that the antimicrobial treatment is ineffective when the SD BDV is greater than 0.46. Also included are methods of treating a subject in need of antimicrobial treatment.

### CROSS-REFERENCE TO RELATED APPLICATIONS

This application claims priority to U.S. Provisional Application 62/764,874 filed on Aug. 16, 2018, which is incorporated herein by reference in its entirety.

### FIELD OF THE DISCLOSURE

The present disclosure is related markers for determining the efficacy of antimicrobials, and novel methods for administering antibiotics, particularly to subjects in a surgical ICU setting.

### BACKGROUND

Infections remain a leading cause of morbidity and mortality in the intensive care unit (ICU). In the United States, infectious complications develop in up to 28% of patients admitted to the ICU and require mechanical ventilation. The current standard of care for detecting infections relies heavily on the physician to make decisions bedside, monitoring trending vitals signs and laboratory work. Hospital mortality for severe infection ranges from 18% to 28% in adults and remains the leading cause of death in adult surgical ICU patients. Assessing the efficacy of antimicrobial treatment in acute systemic inflammation and severe infection, is vital to improved patient outcomes. Moreover, unnecessary empiric antibiotic treatment is undesirable because of the risk of antibiotic-resistant bacterial strains.

A noninvasive, non-doping, rapid stable isotope method to discern the onset of the catabolic state by detecting isotopic changes in the exhaled CO₂ in breath was described in issued U.S. Pat. No. 5,912,178 (the '178 patent). The relative health of an organism was determined by comparing the sampled ratio (C¹³:C¹²) to a baseline ratio in the organism by testing breath samples in a mass spectrometer, for example. The methods disclosed in the '178 patent allow for a non-invasive determination of net catabolic processes of organisms experiencing altered organ function or a deficit in nutrient intake. One disadvantage to the method disclosed in the '178 patent is that a comparison specimen is required to determine if the organism from which a breath sample is measured is in a catabolic state.

Similarly, in U.S. Pat. No. 7,465,276 (the '276 patent), the relative amounts of first and second breath isotopes are measured over time to determine if an organism is experiencing a viral or bacterial infection. Advantages of the method of the '276 patent are that breath samples from an isotopically unenriched organism can be monitored for changes in isotope ratios over time to determine if the organism is experiencing a bacterial or viral infection. A disadvantage of the method is that a baseline measurement from the healthy subject is preferred so that changes from the baseline can be measured that are indicative of infection. In addition, it is generally advisable to obtain measurements over several hours or even several days so that the change in isotope ratio from the baseline ratio can be determined. Thus, determining the transition from a healthy to an infected organism within the short-term infection period, e.g., 30 minutes to 2 hours, may not be possible as the change in slope may not be measurable in this time period.

In addition, U.S. Pat. No. 8,512,676 describes the use of oscillation modes in breath isotope ratio data to identify an “unhealthy” state in an organism. Changes in the frequency and/or amplitude of the oscillation modes can be correlated with the health of an individual. Advantageously, advances in cavity ringdown spectrometry allow for the continuous collection of breath isotope data which permits the identification of oscillatory patterns within the breath isotope data. The identified oscillation modes are particularly useful in determining the transition from a healthy to an infected state in an organism within the short-term infection period, e.g., 30 minutes to 2 hours.

What is needed are improved markers for the response of subjects to antimicrobial treatment, particularly acute surgical and trauma subjects admitted to the ICU.

### BRIEF SUMMARY

In one aspect, a method of determining efficacy of an antimicrobial treatment in a subject comprises

calculating a breath delta value (BDV) for each of at least six breath samples acquired from the subject over a 24 hour period starting from when the subject has been administered the antimicrobial treatment, wherein BDV is determined according to

$$BDV = {δ}^{13} C = \frac{( {}^{13} C / {}^{12} C sample - {}^{13} C / {}^{12} C PDB )}{{}^{13} C / {}^{12} C PDB} \times 1000$$

wherein PDB is a Pee Dee Belemnite reference standard, and BDV is expressed as parts per mil (‰);

calculating a mean standard deviation of BDV (SD BDV) across the six or more breath samples; and
determining that the antimicrobial treatment is effective when the SD BDV is less than or equal to 0.46, or
determining that the antimicrobial treatment is ineffective when the SD BDV is greater than 0.46.

In another aspect, a method of determining efficacy of an antimicrobial treatment in a subject comprises

calculating a breath delta value (BDV) for each of at least six breath samples acquired from the subject over a 24 hour period starting before the subject has been administered the antimicrobial treatment, and calculating a breath delta value (BDV) for each of at least six breath samples acquired from the subject over a 24 hour period starting from when the subject has been administered the antimicrobial treatment, wherein BDV is determined according to

$$BDV = {δ}^{13} C = \frac{( {}^{13} C / {}^{12} C sample - {}^{13} C / {}^{12} C PDB )}{{}^{13} C / {}^{12} C PDB} \times 1000$$

wherein PDB is a Pee Dee Belemnite reference standard, and BDV is expressed as parts per mil (‰);

calculating a mean standard deviation of BDV (SD BDV) across the six or more samples acquired before the subject has been administered the antimicrobial treatment, calculating an SD BDV across the six or more samples acquired after the subject has been administered the antimicrobial treatment, and calculating a % decrease in BDV by subtracting the SD BDV after the antimicrobial treatment from the SD BDV before the antimicrobial treatment; and
determining that the antimicrobial treatment is effective when the % decrease in BDV is greater than or equal to 34%, or
determining that the antimicrobial treatment is ineffective when the % decrease in BDV is less than 34%.

In yet another aspect, a method of treating a subject in need of antimicrobial treatment comprises

administering an initial antimicrobial treatment to the subject;

acquiring at least six breath samples from the subject over a 24 hour period starting from when the subject has been administered the initial antimicrobial treatment;

calculating a breath delta value (BDV) for each of the breath samples according to

$$BDV = {δ}^{13} C = \frac{( {}^{13} C / {}^{12} C sample - {}^{13} C / {}^{12} C PDB )}{{}^{13} C / {}^{12} C PDB} \times 1000$$

wherein PDB is a Pee Dee Belemnite reference standard, and BDV is expressed as parts per mil (‰);

calculating a mean standard deviation of BDV (SD BDV) across the six or more samples; and
determining that the initial antimicrobial treatment is effective when the SD BDV is less than or equal to 0.46, and continuing administering the initial antimicrobial treatment, or
determining that the antimicrobial treatment is ineffective when the SD BDV is greater than 0.46, discontinuing the initial antimicrobial treatment, and administering a subsequent antimicrobial treatment, or adding a subsequent antimicrobial treatment to the antimicrobial treatment.

In a further aspect, a method of treating a subject in need of antimicrobial treatment comprises

acquiring at least six breath samples from the subject over a 24 hour period;

administering an initial antimicrobial treatment to the subject;

acquiring at least six breath samples from the subject over a 24 hour period starting from when the subject has been administered the initial antimicrobial treatment;

calculating a breath delta value (BDV) for each of the breath samples acquired before and after the initial antimicrobial treatment according to

$$BDV = {δ}^{13} C = \frac{( {}^{13} C / {}^{12} C sample - {}^{13} C / {}^{12} C PDB )}{{}^{13} C / {}^{12} C PDB} \times 1000$$

wherein PDB is a Pee Dee Belemnite reference standard, and BDV is expressed as parts per mil (‰);

calculating a mean standard deviation of BDV (SD BDV) across the six or more samples acquired before the subject has been administered the initial antimicrobial treatment, calculating an SD BDV across the six or more samples acquired after the subject has been administered the initial antimicrobial treatment, and calculating a % decrease in BDV by subtracting the SD BDV after the initial antimicrobial treatment from the SD BDV before the initial antimicrobial treatment; and
determining that the antimicrobial treatment is effective when the % decrease in BDV is greater than or equal to 34%, and continuing administering the initial antimicrobial treatment, or
   determining that the antimicrobial treatment is ineffective when the % decrease in BDV is less than 34%, discontinuing the initial antimicrobial treatment, and administering a subsequent antimicrobial treatment, or adding a subsequent antimicrobial treatment to the antimicrobial treatment.

### BRIEF DESCRIPTION OF THE DRAWINGS

FIG. 1 depicts simulated data illustrating the interpretation of BDV from pre-infection through severe infection. (A) Prior to infection, the baseline BDV variance is typically less than 0.4‰ and is not more than 1.4‰ from the mean (left “Normal variance”). (B) As infection sets in and causes initiation of the body wide acute phase response, which can be pre-symptomatic, the BDV decreases (“Onset trend”) as muscle proteins are utilized for both acute phase protein synthesis and metabolic energy needs. (C) As the infection continues to progress, there is an anaerobic shift in metabolism that rapidly drives the BDV in positive direction. An untreated, or improperly treated, infection will exhibit increased variance (of 0.55‰ or more) due to the competing isotopic mechanisms. (D) When appropriate antimicrobial treatments are administered the variance in BDV normalizes.

FIG. 2 shows a Consort Flow Diagram for study enrollment for Example 1. Subjects were enrolled when admitted to the ICU. The subjects were 90% blunt trauma and 10% post-operative in both the infection and non-infection groups.

FIG. 3 shows the variation in BDV after ICU admission, infection or antibiotic treatment. ICU admitted non-infection subjects did not develop infections during the study (n=9). The untreated infection represents the variation in BDV before antibiotic administration in subjects that developed infections (n=11). The average 6 sample standard deviation was calculated by determining the variation for each sample with the 5 preceding samples, then averaging the variation for each timepoint for each subject then taking the square root of the variation resulting in the average standard deviation. The treated infection group represents the standard deviation in BDV for the 6 samples immediately following administration of an antibiotic treatment (n=11). Error bars represent the standard error of the mean, and differing letter super scripts represent significant differences between groups. Statistical analysis was performed using analysis of variance (ANOVA) approach with least significant differences post-hoc analysis. Differences were considered significant with a p-value of less than 0.05.

FIGS. 4-13 show the variation in BDV over time for individual subjects.

FIG. 14 shows a representative example of a subject who did not develop an infection. White blood cell count and core body temperature remained in the normal range per the SIRS definition for the duration of the study. C-reactive protein (CRP) and procalcitonin (PCT) remained elevated with an average value of 16.9(1.2) and 0.47(0.06) respectively. Nutritionally, the subject was NPO (nil per os; unfed) for the first five days of the study and was transitioned to a liquid diet on day 6. In this subject, the BDV remains within 1‰ of the baseline sample for the duration of the study. The mean variance for this subject was 0.28‰.

FIG. 15 shows an example of a subject who developed an infection within the first day of study enrollment. Body temperature spiked out of the normal range each day of the study except day seven and ten with a peak temperature of 38.9 C on day one. White blood cell count (WBC) was out of the normal range every day of the study and ranged from 14.2 to 22.4 cell/mm³. CRP concentration ranged from 3.7 mg/dL on day one to a peak of 7.9 mg/dL on day four with a low of 2.4 mg/dL on day seven. PCT peaked on day one of the study with a value of 0.25 ng/dL and fell to a low value of 0.08 ng/dL on day seven. Initial blood and BAL cultures were negative on days one and two, but subsequent bronchioalveolar lavage (BAL) cultures were positive for Aspergillus fumigatus on day seven. The subject was given Cefuroxime from day one to day eight of the study for surgical site infection prophylaxis, but when cultures reported positive with a fungal infection on day seven cefepime was added. Cefepime treatment was continued beyond the end of the study. The progression trend is evident on days 1 through 7 despite cefuroxime treatment with a mean variation of 0.72‰, but reduced variation of 0.37‰ after cefepime indicates response to treatment.

FIG. 16 shows an example wherein a subject developed an infection and appropriate treatment was administered. The initially high WBC (13.1 k cells/mm3) fell into the normal range on days three through five when it increased to a peak of 15.2 k cells/mm3 on day seven. Body temperatures spiked daily from day three to eight with a peak of 40 C on day six. Blood and BAL cultures confirm infection on day six and Vancomycin and cefepime are administered. The CRP remained between 8.1 to 12.0 mg/dL on days one through six when it went up to 15.2 on day seven. The initially high PCT (4.94 ng/mL) fell from day one to six, when it spiked to 8.81 ng/mL on day seven. While the BDV infection onset trend was unclear in this case, the progression trend is evident in the increasing BDV and the variability of the BDV on days 2 through 4 with a mean variance of 0.51‰. Antibiotic treatment was begun on 5 of the study and the BDV variability decreases to 0.15‰, indicating successful antibiotic treatment.

The above-described and other features will be appreciated and understood by those skilled in the art from the following detailed description, drawings, and appended claims.

### DETAILED DESCRIPTION

Unexpectedly, it is shown herein that breath delta value (BDV) is a marker for the response to antimicrobial treatment, such as in acute surgical and trauma subjects admitted to the ICU. Specifically, the variation in BDV of infected subjects is lower after appropriate antimicrobial treatment than after ineffective or incorrect antimicrobial treatment.

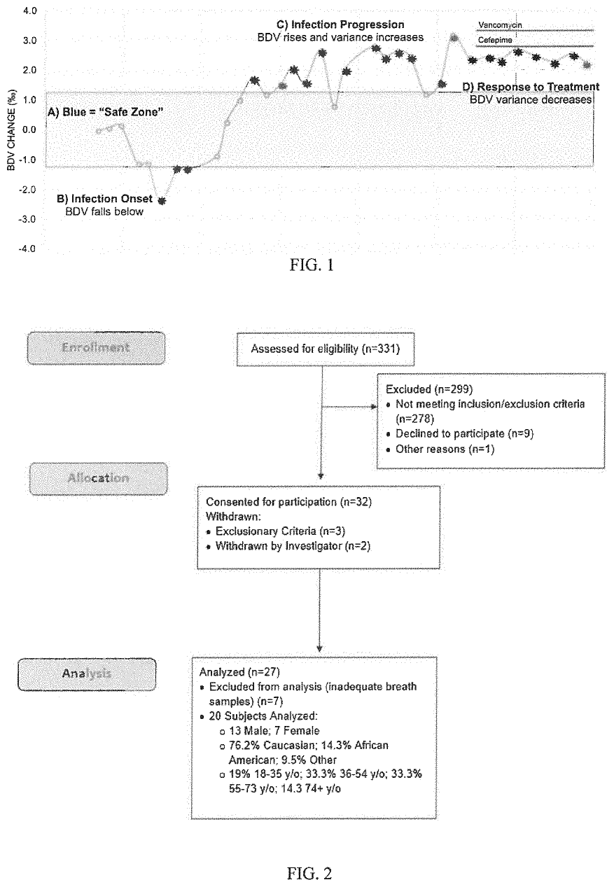

During the early onset of the acute phase response to trauma, the BDV is inversely related to the severity of trauma and, to a greater extent, the presence of a developing infection. FIG. 1 illustrates simulation of BDV from pre-infection therough severe infection and response to treatment. Pre-formed tumor necrosis factor alpha (TNFα) and interleukin-1 (IL-1) induce changes in secondary metabolism. Amino acids are rapidly released from skeletal muscle where they can be used to make acute phase proteins or be metabolized for fuel. Amino acids released during the acute phase response (APR) fractionate based on the molecular weight of the amino acid. This fractionation follows the principles of the kinetic isotope effect. Approximately 1% of the world's stable carbon has the atomic weight of 13 (¹³C); most of the remaining carbon is ¹²C. Amino acids and products that have incorporated ¹³C are less likely to be fully metabolized to CO₂ than amino acids that have incorporated ¹²C, the lighter isotope. The “heavier” carbon amino acids remain as products of the APR (i.e., acute phase proteins) while the “lighter” carbon amino acids are more likely to be completely oxidized to CO₂. The result of this phenomenon is that the ratio of ¹³CO₂ to ¹²CO₂ in breath decreases during the onset of infection (FIG. 1B). However, as the infection progresses, patient metabolism shifts and becomes more anaerobic, and macronutrient metabolism changes from primarily a mixture of carbohydrates and lipids to primarily carbohydrate and body proteins. Due to isotopic discrimination against ¹³C at several steps in their synthesis, lipids are 3-5‰ lighter than carbohydrates or proteins. Thus, a shift in macronutrient oxidation during the progression of infection causes a rapid increase in the ¹³C:¹²C ratio (FIG. 1C). Because of the competing isotopic mechanisms during an untreated infection the BDV has a higher variance than normal (FIG. 1A). After appropriate antimicribial treatment is administered the variance in BDV returns to normal (FIG. 1D).

As used herein, the breath delta value (δ ¹³C) is calculated using the following formula with Pee Dee Belemnite (PDB) as the reference standard.

$$BDV = {δ}^{13} C = \frac{( {}^{13} C / {}^{12} C sample - {}^{13} C / {}^{12} C PDB )}{{}^{13} C / {}^{12} C PDB} \times 1000$$

Data are expressed as delta values in parts per ml (‰).

The standard isotope ratio of PDB is 0.0112372. A positive δ ¹³C occurs when the measured isotope ratio is higher relative to PDB, while a negative δ ¹³C occurs when the measured isotope ratio is lower relative to PDB. Since PDB contains the heaviest known naturally occurring ¹³C/¹²C ratio, all measurements of the natural abundance of carbon isotopes are negative.

In one aspect, breath samples are collected in sample bags such as 1 L Tedlar or metal foil bags. Breath samples can be directly collected into an instrument designed for such collection.

In one embodiment, relative isotope measurements are made using cavity ringdown spectroscopy (CRDS). CRDS uses infrared laser absorption to measure the concentrations of ¹³CO₂ and ¹²CO₂ carbon signals, and reports precise total CO₂ levels as well as the ¹³CO₂/¹²CO₂ ratio. An exemplary instrument is a Picarro G2101-i Isotopic CO₂ analyzer. Other methods to measure breath δ ¹³C include isotope mass spectrometry.

In an aspect, a method of determining efficacy of an antimicrobial treatment in a subject comprises

calculating a breath delta value (BDV) for each of at least six breath samples acquired from the subject over a 24 hour period starting from when the subject has been administered the antimicrobial treatment, wherein BDV is determined according to

$$BDV = {δ}^{13} C = \frac{( {}^{13} C / {}^{12} C sample - {}^{13} C / {}^{12} C PDB )}{{}^{13} C / {}^{12} C PDB} \times 1000$$

wherein PDB is a Pee Dee Belemnite reference standard, and BDV is expressed as parts per mil (‰);

calculating a mean standard deviation of BDV (SD BDV) across the six or more breath samples; and
determining that the antimicrobial treatment is effective when the SD BDV is less than or equal to 0.46, or
determining that the antimicrobial treatment is ineffective when the SD BDV is greater than 0.46.

In another aspect, a method of determining efficacy of an antimicrobial treatment in a subject comprises

calculating a breath delta value (BDV) for each of at least six breath samples acquired from the subject over a 24 hour period starting before the subject has been administered the antimicrobial treatment, and calculating a breath delta value (BDV) for each of at least six breath samples acquired from the subject over a 24 hour period starting from when the subject has been administered the antimicrobial treatment, wherein BDV is determined according to

$$BDV = {δ}^{13} C = \frac{( {}^{13} C / {}^{12} C sample - {}^{13} C / {}^{12} C PDB )}{{}^{13} C / {}^{12} C PDB} \times 1000$$

wherein PDB is a Pee Dee Belemnite reference standard, and BDV is expressed as parts per mil (‰);

calculating a mean standard deviation of BDV (SD BDV) across the six or more samples acquired before the subject has been administered the antimicrobial treatment, calculating an SD BDV across the six or more samples acquired after the subject has been administered the antimicrobial treatment, and calculating a % decrease in BDV by subtracting the SD BDV after the antimicrobial treatment from the SD BDV before the antimicrobial treatment; and
determining that the antimicrobial treatment is effective when the % decrease in BDV is greater than or equal to 34%, or
determining that the antimicrobial treatment is ineffective when the % decrease in BDV is less than 34%.

In the foregoing embodiments, wherein it is determined that the antimicrobial treatment is effective and the antimicrobial treatment may be continued. In the foregoing embodiments, wherein it is determined that that the antimicrobial treatment is ineffective, the antimicrobial treatment may be discontinued and a subsequent antimicrobial treatment may be initiated; or a subsequent antimicrobial treatment may be added to the antimicrobial treatment. For example, the subject may be treated with an antibiotic for a suspected bacterial infection, however, upon determination of a fungal infection, an antifungal may be used in addition to or in place of the antibiotic.

A method of treating a subject in need of antimicrobial treatment comprises administering an initial antimicrobial treatment to the subject;

acquiring at least six breath samples from the subject over a 24 hour period starting from when the subject has been administered the initial antimicrobial treatment;

calculating a breath delta value (BDV) for each of the breath samples according to

$$BDV = {δ}^{13} C = \frac{( {}^{13} C / {}^{12} C sample - {}^{13} C / {}^{12} C PDB )}{{}^{13} C / {}^{12} C PDB} \times 1000$$

wherein PDB is a Pee Dee Belemnite reference standard, and BDV is expressed as parts per mil (‰);

calculating a mean standard deviation of BDV (SD BDV) across the six or more samples; and
determining that the initial antimicrobial treatment is effective when the SD BDV is less than or equal to 0.46, and continuing administering the initial antimicrobial treatment, or
determining that the antimicrobial treatment is ineffective when the SD BDV is greater than 0.46, discontinuing the initial antimicrobial treatment, and administering a subsequent antimicrobial treatment, or adding a subsequent antimicrobial treatment to the antimicrobial treatment.

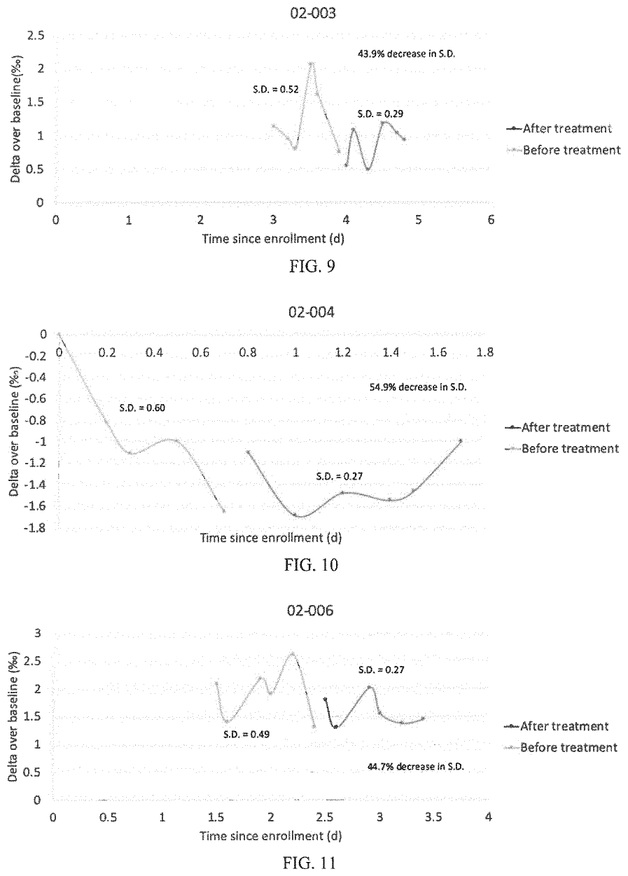

As shown, for example in FIG. 4, a 53.1% decrease in SD of BDV observed after antimicrobial treatment demonstrates that appropriate antimicrobial treatment was administered to this subject. In contrast, as shown in FIG. 5, a 17.7% decrease in SD of BDV observed after antimicrobial treatment demonstrates that appropriate antimicrobial treatment was not administered to this subject.

Exemplary subjects are mammalian subjects, specifically human subjects. In any of the methods described herein, the subject can be an acute surgical or trauma subject admitted to an intensive care unit and suspected of having an infection.

Also in any of the embodiments described herein, the subject meets at least two systemic inflammatory response syndrome criteria, infection is suspected based on diagnostic imaging, or infection is suspected based on culture results. Systemic inflammatory response syndrome criteria include body temperature >38° C. or <36° C., heart rate >90 beats/min, respiratory rate >20 breaths/min, or white blood cell >12,000 cells/mm³ or <4,000 cells/mm³, or >10% immature neutrophils.

In any of the foregoing embodiments, the subject may be intubated.

Exemplary antimicrobials include antibiotics, antifungals, antivirals, and antiparasitics.

Exemplary antibiotics include aztreonam; cefotetan and its disodium salt; loracarbef; cefoxitin and its sodium salt; cefazolin and its sodium salt; cefaclor; ceftibuten and its sodium salt; ceftizoxime; ceftizoxime sodium salt; cefoperazone and its sodium salt; cefuroxime and its sodium salt; cefuroxime axetil; cefprozil; ceftazidime; cefotaxime and its sodium salt; cefadroxil; ceftazidime and its sodium salt; cephalexin; hexachlorophene; cefamandole nafate; cefepime and its hydrochloride, sulfate, and phosphate salt; cefdinir and its sodium salt; ceftriaxone and its sodium salt; cefixime and its sodium salt; cetylpyridinium chloride; ofoxacin; linexolid; temafloxacin; fleroxacin; enoxacin; gemifloxacin; lomefloxacin; astreonam; tosufloxacin; clinafloxacin; cefpodoxime proxetil; chloroxylenol; methylene chloride, iodine and iodophores (povidone-iodine); nitrofurazone; meropenem and its sodium salt; imipenem and its sodium salt; cilastatin and its sodium salt; azithromycin; clarithromycin; dirithromycin; erythromycin and hydrochloride, sulfate, or phosphate salts ethylsuccinate, and stearate forms thereof, clindamycin; clindamycin hydrochloride, sulfate, or phosphate salt; lincomycin and hydrochloride, sulfate, or phosphate salt thereof, tobramycin and its hydrochloride, sulfate, or phosphate salt; streptomycin and its hydrochloride, sulfate, or phosphate salt; vancomycin and its hydrochloride, sulfate, or phosphate salt; neomycin and its hydrochloride, sulfate, or phosphate salt; acetyl sulfisoxazole; colistimethate and its sodium salt; quinupristin; dalfopristin; amoxicillin; ampicillin and its sodium salt; clavulanic acid and its sodium or potassium salt; penicillin G; penicillin G benzathine, or procaine salt; penicillin G sodium or potassium salt; carbenicillin and its disodium or indanyl disodium salt; piperacillin and its sodium salt; .alpha.-terpineol; thymol; taurinamides; nitrofurantoin; silver-sulfadiazine; hexetidine; methenamine; aldehydes; azylic acid; silver; benzyl peroxide; alcohols; carboxylic acids; salts; nafcillin; ticarcillin and its disodium salt; sulbactam and its sodium salt; methylisothiazolone, moxifloxacin; amifloxacin; pefloxacin; nystatin; carbepenems; lipoic acids and its derivatives; beta-lactams antibiotics; monobactams; aminoglycosides; microlides; lincosamides; glycopeptides; tetracyclines; chloramphenicol; quinolones; fucidines; sulfonamides; macrolides; ciprofloxacin; ofloxacin; levofloxacins; teicoplanin; mupirocin; norfloxacin; sparfloxacin; ketolides; polyenes; azoles; penicillins; echinocandines; nalidixic acid; rifamycins; oxalines; streptogramins; lipopeptides; gatifloxacin; trovafloxacin mesylate; alatrofloxacin mesylate; trimethoprims; sulfamethoxazole; demeclocycline and its hydrochloride, sulfate, or phosphate salt; doxycycline and its hydrochloride, sulfate, or phosphate salt; minocycline and its hydrochloride, sulfate, or phosphate salt; tetracycline and its hydrochloride, sulfate, or phosphate salt; oxytetracycline and its hydrochloride, sulfate, or phosphate salt; chlortetracycline and its hydrochloride, sulfate, or phosphate salt; metronidazole; dapsone; atovaquone; rifabutin; linezolide; polymyxin B and its hydrochloride, sulfate, or phosphate salt; sulfacetamide and its sodium salt; clarithromycin; and the like, and combinations comprising at least one of the foregoing.

Exemplary antifungals include amphotericin B; pyrimethamine; flucytosine; caspofungin acetate; fluconazole; griseofulvin; terbinafine and its hydrochloride, sulfate, or phosphate salt; amorolfine; triazoles (Voriconazole); flutrimazole; cilofungin; LY303366 (echinocandines); pneumocandin; imidazoles; omoconazole; terconazole; fluconazole; amphotericin B, nystatin, natamycin, liposomal amptericin B, liposomal nystatins; griseofulvin; BF-796; MTCH 24; BTG-137586; RMP-7/Amphotericin B; pradimicins; benanomicin; ambisome; ABLC; ABCD; Nikkomycin Z; flucytosine; SCH 56592; ER30346; UK 9746; UK 9751; T 8581; LY121019; ketoconazole; micronazole; clotrimazole; econazole; ciclopirox; naftifine; itraconazole; and the like, and combinations comprising at least one of the foregoing.

Exemplary antivirals include Abacavir, Aciclovir, Acyclovir, Adefovir, Amantadine, Amprenavir, Ampligen, Arbidol, Atazanavir, Atripla (fixed dose drug), Boceprevir, Cidofovir, Combivir (fixed dose drug), Darunavir, Delavirdine, Didanosine, Docosanol, Edoxudine, Efavirenz, Emtricitabine, Enfuvirtide, Entecavir, Entry inhibitors, Famciclovir, Fixed dose combination (antiretroviral), Fomivirsen, Fosamprenavir, Foscarnet, Fosfonet, Fusion inhibitor, Ganciclovir, Ibacitabine, Imunovir, Idoxuridine, Imiquimod, Indinavir, Inosine, Integrase inhibitor, Interferon type III, Interferon type II, Interferon type I, Interferon, Lamivudine, Lopinavir, Loviride, Maraviroc, Moroxydine, Methisazone, Nelfinavir, Nevirapine, Nexavir, Nucleoside analogues, Oseltamivir (Tamiflu), Peginterferon alfa-2a, Penciclovir, Peramivir, Pleconaril, Podophyllotoxin, Protease inhibitor (pharmacology), Raltegravir, Reverse transcriptase inhibitor, Ribavirin, Rimantadine, Ritonavir, Pyramidine, Saquinavir, Stavudine, Synergistic enhancer (antiretroviral), Tea tree oil, Tenofovir, Tenofovir disoproxil, Tipranavir, Trifluridine, Trizivir, Tromantadine, Truvada, Valaciclovir (Valtrex), Valganciclovir, Vicriviroc, Vidarabine, Viramidine, Zalcitabine, Zanamivir (Relenza), Zidovudine, and the like, and combinations comprising at least one of the foregoing.

Exemplary antiparasitics include mebendazole, pyrantel pamoate, thiabendazole, diethylcarbamazine, ivermectin, niclosamide, praziquantel, albendazole, rifampin, amphotericin B, melarsoprol, eflornithine, metronidazole, tinidazole, miltefosine, and the like, and combinations comprising at least one of the foregoing.

The invention is further illustrated by the following non-limiting examples.

### Methods

Study Design:

The study was conducted as a multi-center prospective study, at four academic research hospitals, to assess exhaled ¹³CO₂/¹²CO₂ BDV as an indicator of infection in 20 critically ill and injured adult ICU subjects. Critically ill adult ICU subjects who were not suspected of having an infection at the time of ICU admission were enrolled as study subjects.

Inclusion Criteria:
1) age 18 years or older;
2) critically ill patient admitted to the ICU;
3) enrolled within 48 hours of ICU admittance;
4) expected duration of hospital stay at least 120 hours (five days) from time of study enrollment; and
5) subject or legally authorized representative speaks a language of which the IRB has approved a consent form.

Exclusion Criteria:
1) known or suspected infection at time of enrollment;
2) known use of systemic antibiotic, antimicrobial and/or antifungal therapy within the seven days prior to hospital admission;
3) currently active cancer, defined as receiving treatment or intend to receive treatment within hospital stay for cancer (including but not limited to: radiation, chemotherapy, systemic orals, etc.);
4) if not intubated, unable to cooperate with providing a breath sample;
5) expected death within 24 hours of enrollment or lack of commitment to aggressive treatment by family/medical team (e.g., likely to withdraw life support measures within 24 hrs of screening);
6) female who was pregnant or lactating (negative serum or urine pregnancy test results within 48 hours of enrollment or to be performed during screening);
7) prisoner;
8) known participation in an investigational and interventional research study within 30 days prior to enrollment;
9) individuals who were directly affiliated with sponsor or study staff, or their immediate families; and
10) any patient that was deemed unfit for study participation, per the Investigator's discretion.

Study team members conducted the informed consent discussion with the potential subject or surrogate in a location where a private conversation could be held. The study team member explained the study procedures, the purpose of the study, and that treatment of the potential subject is not the purpose of the study. Coercion was prevented by stressing that the potential subject or surrogate does not have to agree to participate, and that the care of the potential subject will not be affected by the decision to participate.

Exhaled breath samples were collected upon subject enrollment and every four hours thereafter until the subject was discharged home, transferred to a general care unit/status, or after seven days of breath sample collection, whichever came first. Each sample was collected in four-hour intervals, calculated from the initial sample time, with a window of ±1 hour. Samples collected outside of the specified time interval were still analyzed.

For mechanically ventilated subjects, an appropriately trained and qualified member of the subject's clinical care team or respiratory team obtained expired breath from a side port adaptor in the expiratory limb of the subject's breathing circuit. The breath sample was captured in a small gas tight sample bag. Sample collection did not interfere with operation of the mechanical ventilator or breathing circuit. The breath sample was collected over approximately 2-4 breath cycles. Non-mechanically ventilated subjects were asked to provide a sample by exhaling into the sample bag. If the subject was not ventilated but had difficulty inflating a bag using the supplied mouthpiece, a mask collection option was made available. Collecting a sample using the mask was performed by attaching a sample bag to the breathing mask via a connector. The mask was placed over the subject's nose and mouth during exhalation.

An endpoint adjudication committee (EAC), composed of three independent senior infectious disease experts, not involved in the subject clinical care, reviewed each study subject's data to determine the clinical time and date of infection. Each EAC member independently reviewed the subject cases and completed the EAC Infection Status case report form. Further, the EAC met as a group to discuss each subject's infection status and make a majority decision. The group decision was based on the individual reports, with a two-thirds majority needed to determine status. In cases where an infection developed, the EAC placed a time and date stamp for time of first suspicion of infection and confirmation based on clinical judgement, culture, or diagnostic imaging. Subjects were considered to be ‘suspected of infection’ if they met at least two systemic inflammatory response syndrome (SIRS) criteria (i.e., body temperature >38° C. or <36° C., heart rate >90 beats/min, respiratory rate >20 breaths/min, or white blood cell >12,000 cells/mm³ or <4,000 cells/mm³, or >10% immature neutrophils), and were given antibiotics, and/or diagnostic imaging or cultures were ordered. For statistical analysis, subjects categorized by the EAC as ‘no suspicion’ or ‘low suspicion of infection’ were grouped and considered to have no infection, and subjects categorized as ‘high suspicion of infection’ or ‘overt infection’ were grouped and considered to have developed an infection.

Breath Sample Analysis:

Breath samples were shipped to a central laboratory (Isomark, LLC, Madison, Wis.) within 48 hours of final collection from each subject. The breath samples were analyzed using the Canary™ device. The ¹³CO₂/¹²CO₂ ratio of each sample was determined from direct measurement and calculated using Pee Dee Belemnite (PDB) as a standard reference:

$$BDV = {δ}^{13} {CO}_{2} = \frac{( {}_{}^{13}{CO}{}_{2}^{} / {}_{}^{12}{CO}{}_{2}^{} sample - {}_{}^{13}{CO}{}_{2}^{} / {}_{}^{12}{CO}{}_{2}^{} PDB )}{{}_{}^{13}{CO}{}_{2}^{} / {}_{}^{12}{CO}{}_{2}^{} PDB} \times 1000$$

where BDV is expressed as parts per mil (‰). Each subject was used as their own control for the purpose of trend analysis and the first breath sample collected was considered the “baseline” sample. The delta over baseline (DOB) was then calculated by subtracting the baseline sample from subsequent samples as described:

DOB=BDVₛₐₘₚₗₑ−BDV_{baseline}

DOB values for subjects with and without infection were graphed as a function of time, and the standard error of the mean for each time point was calculated and graphed as error bars.

Statistical Analysis:

The standard deviation in BDV for the uninfected and untreated infection groups was calculated by first finding the mean variance for any 6 consecutive samples, then calculating the standard deviation by finding the square root of the 6 sample variance mean. For the treated infection group the standard deviation was calculated using only the 6 samples before or after administration of antimicrobial treatment. Response to treatment was assessed by computing the percent change in the standard deviation of the six samples prior to treatment with the standard deviation of the six samples after treatment for each subject who received antimicrobial treatment for infection. To determine differences in the mean standard deviation between groups an ANOVA analysis with least significant differences post-hoc analysis was used. Differences were considered significant if p<0.05.

Sample Size Determination:

From preliminary data collected, a sustained change in BDV of 1.0‰ or more was estimated to correlate with the onset of infection when the subject was used as his-her control. The average intra-subject standard deviation (SD) across time points in critically ill adult subjects previously studied was 0.95‰, regardless of the underlying medical conditions. The inter-subject SD was expected to be 1.0‰ at most. Using the BDV measurements during the breath sample monitoring period (breath sample assessments every four hours), the expected overall SD of the mean BDV measurements (across time points) was less than 1.0‰. Furthermore, during the monitoring period, the mean difference in the BDV between subjects who were diagnosed with an infection and subjects not diagnosed with an infection was expected to be 1.0‰ or more (primary hypothesis). Assuming an overall standard deviation of 1.0‰, a total sample size at least eight infections was required for 80% power to detect a difference of 1.0‰ with a two-sided p-value of less than 0.05.

### Results:

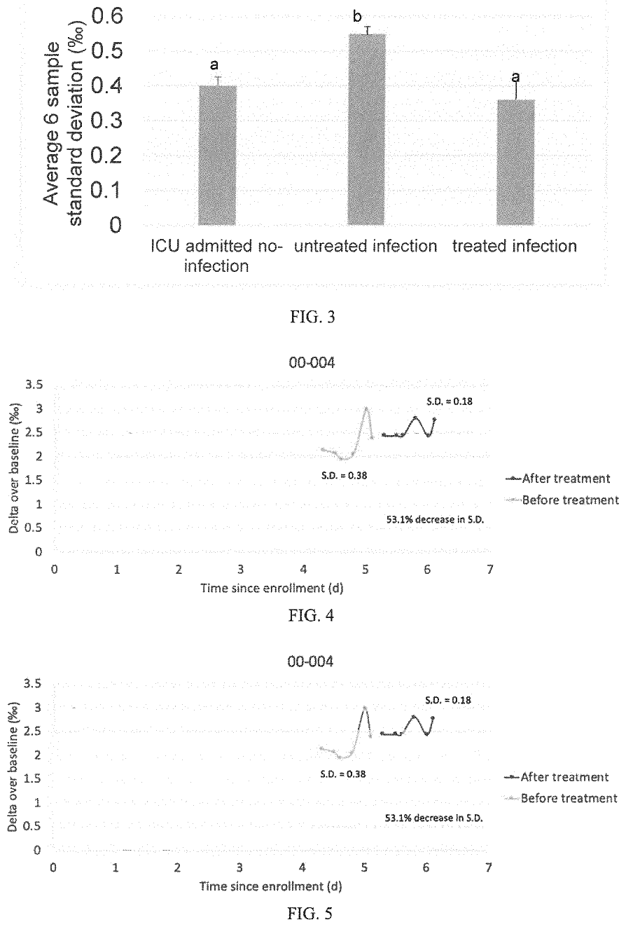

During the study, 32 subjects were consented for participation after acute trauma or surgery. Three of the consented subjects were withdrawn due to meeting exclusion criteria, and two were withdrawn by the investigator (FIG. 2). Seven of the analyzed subjects were excluded from analysis due to inadequate breath sample collection (FIG. 2). Of the seven excluded subjects, five were discharged in less than five days (exclusion criteria), one experienced agitation and refused to provide samples, and in one case the sample collection device was mistakenly placed on the inspiratory limb of the ventilator instead of the expiratory limb (user error). Table 1 lists the ICU admitting diagnosis for each subject, and the evidence of infection used to classify each subject as infected or non-infected. Of the 20 subjects included in the analysis, infections developed in 11 of the subjects, one of which was on antimicrobial treatment before enrollment. Since each subject was used as their own control for the BDV measurement, the delta over baseline calculation was used for breath measurements.

TABLE 1
Clinical diagnostics for each subject analyzed in the ‘infection’ group based on
the EAC review. Of the 9 non-infected, 8 were admitted as ‘Trauma- Blunt’, and 1 as ‘Post-op’.

| | | Suspected | Confirmed | Infection | Detailed results of Infection |
|---|---|---|---|---|---|
| | ICU | Infection | Infection | Category/ | HR (bpm), Temp (degrees celsius), |
| | Admitting | (Hrs since | (Hrs since | Diagnostic | RR (breaths/min) |
| Subject | Diagnosis | enrollment) | enrollment) | mechanism | WBC (kcells/uL), Bands (%) |
| 1 | Trauma- | 11.5 | 128.0 | High | HR: 121 Temp: 39.5 RR: 28 WBC: |
| | Blunt | | | Suspicion | 10.3 (75% Bands) |
| | | | | of Infection | Culture: BAL- S. aureus & Candida sp, |
| | | | | Culture | endogenous |
| | | | | Proven | Xray: no infection suspected, both |
| | | | | | atalectasis |
| | | | | | Vancomycin and Piperacillin- |
| | | | | | Tazobactam |
| 2 | Trauma- | 43.2 | 131.2 | High | HR: 93 Temp: 38.8 RR: 33 WBC: 14.2 |
| | Blunt | | | Suspicion | (81% bands) |
| | | | | of Infection | Culture: BAL- No growth present |
| | | | | Clinical | Xray: no infection, both atelectasis, |
| | | | | Suspicion | both effusion |
| | | | | | Metronidozole and Vancomycin |
| 3 | Trauma- | 75.5 | 103.8 | Overt | HR: 129 Temp 40.0 RR: 31 WBC: 12.1 |
| | Blunt | | | Infection | (80% Bands) |
| | | | | Culture | Culture: BAL- Strep. constellatu & |
| | | | | Proven | Staph. Aureus |
| | | | | | Xray: no infection, R atalectasis, R |
| | | | | | effusion, bilat pneumothorax |
| | | | | | Vancomycin and Cefepime |
| 4 | Trauma- | 4.0 | 14.0 | Overt | HR: 122 Temp: 38.4 RR: 26 WBC: |
| | Blunt | | | Infection | 10.8 (76% Bands) |
| | | | | Culture | Culture: BAL- Haemophilius influenza |
| | | | | Proven | & Staph. Aureus, endogenous flora |
| | | | | | Xray: Infection suspected- |
| | | | | | subcutaneous emphysema, R |
| | | | | | atalectasis, L chest tube, L contusion |
| | | | | | Ciprofloxacin, Vancomycin and |
| | | | | | Cefepime |
| 5 | Trauma- | 0.0 | 131.4 | Overt | HR: 108 Temp: 38.3 RR: 27 WBC: 21.2 |
| | Blunt | | | Infection | (52% Bands) |
| | | | | Culture | Culture: Fungal with smear- apergillus |
| | | | | Proven | fuigatus BAL- acinetobacter baumanii, |
| | | | | | klebsiella oxytoca, endogenous flora |
| | | | | | Xray: no infection, Bilat atelectasis, R |
| | | | | | effusion |
| | | | | | Cefepime |
| 6 | Trauma- | 88.2 | 152.3 | Overt | HR: 113 Temp: 38.4 RR: 35 WBC: |
| | Blunt | | | Infection | 14.1 (Bands not reported) |
| | | | | Culture | Culture: BAL- P. aeruginosa |
| | | | | Proven | (>100,000) & E. coli (11,000) |
| | | | | | Cefepime |
| 7 | Trauma- | 43.3 | 60.2 | Overt | HR: 139 Temp: 39.4 RR: 27 WBC: |
| | Blunt | | | Infection | 10.2 (Bands not reported) |
| | | | | Culture | Culture: Urine- enterococcus, BAL- |
| | | | | Proven | gram negative rods, coccobacilli |
| | | | | | (haemophilos influenzae) |
| | | | | | Ceftriaxone Rocephin |
| 8 | Trauma- | 24.4 | 61.3 | Overt | HR: 151 Temp: 37.7 RR: 36 WBC: 8.9 |
| | Unk | | | Infection | (Bands not reported) |
| | | | | Culture | Culture: BAL-Pseudomonas |
| | | | | Proven | aeruginosa > 100,000 |
| | | | | | Xray: increased conspicuity of airspace |
| | | | | | opacities within the R perihilar and R |
| | | | | | upper lobe regions |
| | | | | | Cefepime |
| 9 | Trauma- | 3.3 | 20.4 | Overt | HR: 101 Temp: 38.6 RR: 26 WBC: |
| | Blunt | | | Infection | 13.9 (Bands not reported) |
| | | | | Culture | Culture: BAL- Gram Negative > 100,000 |
| | | | | Proven | Serratia marcescens |
| | | | | | Xray: Pulmonary Interstitial Edema |
| 10 | Post-Op | 124.0 | 133.8 | High | HR: 123 Temp: 37.1 RR: 19 WBC: |
| | | | | Suspicion | 20.4 (Bands not reported) |
| | | | | of Infection | CT Abd/Pelv: 7 cm fluid collection |
| | | | | CT Proven | within the mesorectal region which |
| | | | | | extend to presacral |
| | | | | | Metronidazole and Ciprofloxacin |
| 11 | Trauma- | 78.5 | 105.4 | Overt | HR: 113 Temp: 39.5 RR: 30 WBC: 14.8 |
| | Blunt | | | Infection | (Bands not reported) |
| | | | | Culture | Culture: BAL- MRSA ≥ 100,000/mL |
| | | | | Proven | colonies, citrobacter koseri ≥ 100,000 |
| | | | | | colonies/mL |
| | | | | | Xray: Possible aspiration pneumonia, |
| | | | | | possible cavitary pneumonia |

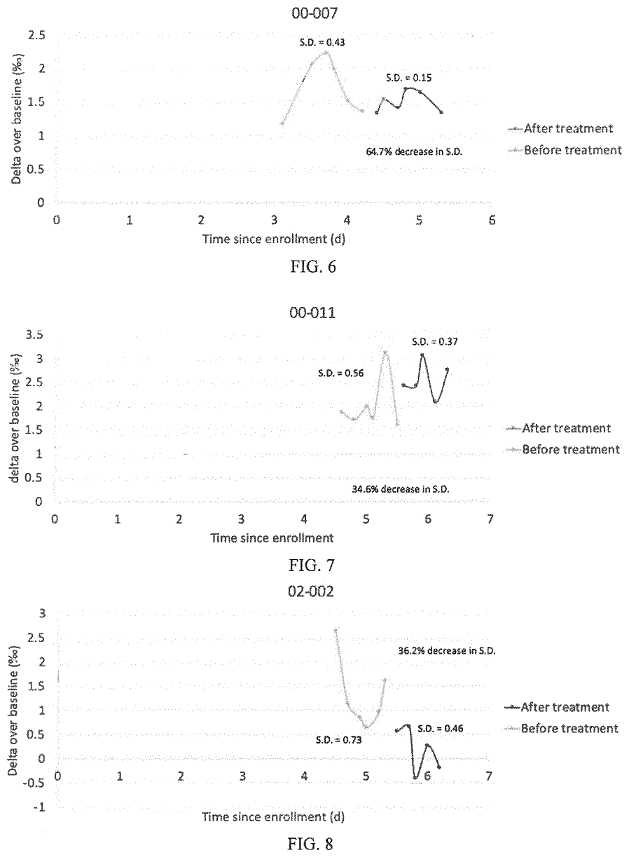

The variation in the BDV was examined separately in subjects without infection, with infection prior to appropriate treatment, and following antimicrobial treatment. The mean standard deviation in subjects without infection was 0.40‰±0.02, while it was 0.55‰±0.02, significantly higher, in subjects with an untreated infection. The mean standard deviation in infected subjects treated with appropriate antimicrobials was 0.36‰±0.05 and similar to uninfected subjects and significantly lower than in subjects with untreated infection (FIG. 3). For each individual who developed an infection during the study, the standard deviation was calculated for the six samples before and after administration of appropriate antibiotics, and the percent change in standard deviation after treatment was calculated. The mean percent decrease after antimicrobial treatment was 44.3%±5.5.

FIGS. 4-13 show graphic representations of the change in standard deviation after antimicrobial administration for ten individuals.

For the individual in FIG. 4, the SD of BDV was 0.38, and after antibiotic treatment the SD was 0.18. A 53.1% decrease in SD of BDV was observed after antimicrobial treatment. Thus, the variation in BDV before and after antimicrobial treatment shows that appropriate antimicrobial treatment was administered to this subject.

For the individual in FIG. 5, the SD of BDV was 0.76, and after antibiotic treatment the SD was 0.62. Only a 17.7% decrease in SD of BDV was observed after antimicrobial treatment. Thus, the variation in BDV before and after antimicrobial treatment shows that appropriate antimicrobial treatment was not administered to this subject.

For the individual in FIG. 6, the SD of BDV was 0.43, and after antibiotic treatment the SD was 0.15. A 64.7% decrease in SD of BDV was observed after antimicrobial treatment. Thus, the variation in BDV before and after antimicrobial treatment shows that appropriate antimicrobial treatment was administered to this subject.

For the individual in FIG. 7, the SD of BDV was 0.56, and after antibiotic treatment the SD was 0.37. A 34.6% decrease in SD of BDV was observed after antimicrobial treatment. Thus, the variation in BDV before and after antimicrobial treatment shows that appropriate antimicrobial treatment was administered to this subject.

For the individual in FIG. 8, the SD of BDV was 0.73, and after antibiotic treatment the SD was 0.46. A 36.2% decrease in SD of BDV was observed after antimicrobial treatment. Thus, the variation in BDV before and after antimicrobial treatment shows that appropriate antimicrobial treatment was administered to this subject. Note that only 5 breath samples were taken post-treatment, and at least 6 samples is preferred.

For the individual in FIG. 9, the SD of BDV was 0.52, and after antibiotic treatment the SD was 0.29. A 43.9% decrease in SD of BDV was observed after antimicrobial treatment. Thus, the variation in BDV before and after antimicrobial treatment shows that appropriate antimicrobial treatment was administered to this subject.

For the individual in FIG. 10, the SD of BDV was 0.60, and after antibiotic treatment the SD was 0.27. A 54.9% decrease in SD of BDV was observed after antimicrobial treatment. Thus, the variation in BDV before and after antimicrobial treatment shows that appropriate antimicrobial treatment was administered to this subject.

For the individual in FIG. 11, the SD of BDV was 0.49, and after antibiotic treatment the SD was 0.27. A 44.7% decrease in SD of BDV was observed after antimicrobial treatment. Thus, the variation in BDV before and after antimicrobial treatment shows that appropriate antimicrobial treatment was administered to this subject.

For the individual in FIG. 12, the SD of BDV was 0.51, and after antibiotic treatment the SD was 0.40. A 21.8% decrease in SD of BDV was observed after antimicrobial treatment. Without being held to theory it is believed that appropriate antimicrobial treatment was not administered to this subject. In this case, the infection was a localized surgical site infection which did cause a dramatic increase in white blood cell count at the time of clinical diagnosis that did not resolve for at least 4 days. The localized infection may have caused lower SD of BDV than would be expected with a more severe infection. The un-resolving clinical signs (i.e. abnormal white blood cell count) indicate the treatment was not improving the infection. These reasons may account for the discrepancy with the other cases.

For the individual in FIG. 13, the SD of BDV was 0.86, and after antibiotic treatment the SD was 0.24. A 71.7% decrease in SD of BDV was observed after antimicrobial treatment. Thus, the variation in BDV before and after antimicrobial treatment shows that appropriate antimicrobial treatment was administered to this subject.

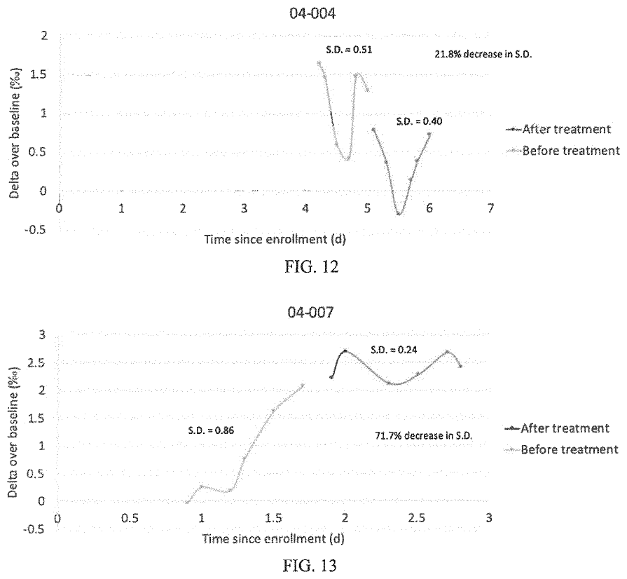

FIG. 14 is a representative example of a subject who did not develop an infection. White blood cell count and core body temperature remained in the normal range per the SIRS definition for the duration of the study. CRP and PCT remained elevated with an average value of 16.9 mg/dL±1.2 and 0.47 ng/mL±0.06 respectively. Nutritionally, this subject was nil per os (NPO; withholding foods and liquids) for the first five days of the study and was transitioned to liquids on day 6. In this subject, the BDV remained within 1‰ of the baseline sample for the duration of the study. The mean variance for this subject was 0.28‰.

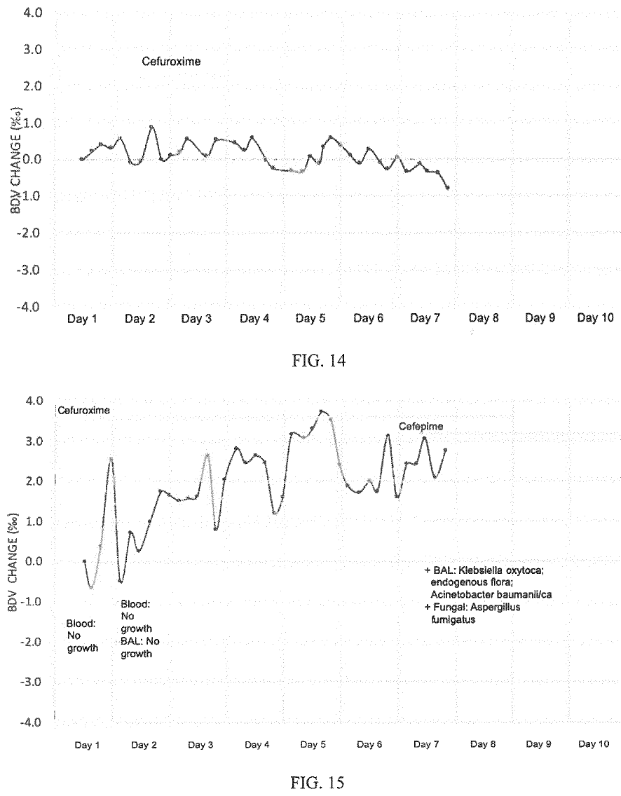

FIG. 15 is a representative example of a subject who developed an infection within the first day of study enrollment. Body temperature spiked out of the normal range each day of the study except days seven and ten with a peak temperature of 38.9° C. on day one. White blood cell count (WBC) was out of the normal range every day of the study and ranged from 14.2 to 22.4 kcells/mm³. CRP concentration ranged from 3.7 mg/dL on day one to a peak of 7.9 mg/dL on day four with a low of 2.4 mg/dL on day seven. PCT peaked on day one of the study with a value of 0.25 ng/dL and fell to a low value of 0.08 ng/dL on day seven. Initial blood and bronchioalveolar (BAL) cultures were negative on days one and two, but subsequent BAL cultures were positive for Aspergillus fumigatus on day seven. The subject was given Cefuroxime from day one to day eight of the study for surgical site infection prophylaxis, but when cultures reported positive with a fungal infection on day seven cefepime was added. Cefepime treatment was continued beyond the end of the study. The progression trend was evident on days 1 through 7 despite cefuroxime treatment with a mean variation of 0.72‰, but reduced variation of 0.37‰ after cefepime initiation, indicating response to treatment.

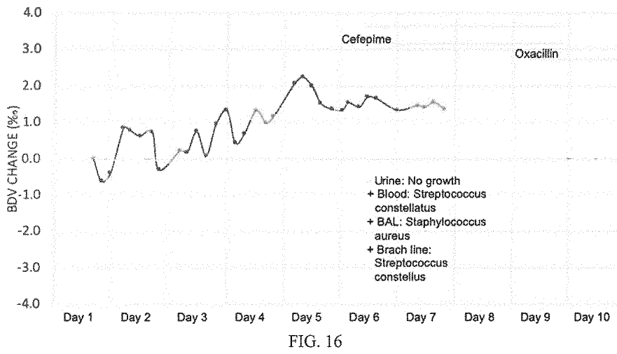

FIG. 16 is a representative example of a subject who developed an infection and appropriate treatment was administered. The initially high WBC (13.1 kcells/mm³) fell into the normal range on days three through five, then increased to a peak of 15.2 kcells/mm³ on day seven. Body temperatures spiked daily from day three to eight with a peak of 40° C. on day six. Blood and BAL cultures confirmed infection on day six and Vancomycin and cefepime are administered. The CRP remained between 8.1 to 12.0 mg/dL on days one through six when it went up to 15.2 on day seven. The initially high PCT (4.94 ng/mL) fell from day one to six, when it spiked to 8.81 ng/mL on day seven. While the BDV infection onset trend was unclear in this case, the progression trend was evident in the increasing BDV and the variability of the BDV on days 2 through 4 with a mean variance of 0.51‰. Antibiotic treatment began on day 5 of the study and the BDV variability decreased to 0.15‰, indicating successful antibiotic treatment.

### Discussion

This study was a pilot investigation using BDV technology as an adjunct for assessing the response to antimicrobial treatment in critically ill trauma and surgical patients with infections. We demonstrated a method that is can quickly determine if antimicrobial treatments are effective by assessing variation in the BDV.

The variation in BDV over time may be a valuable tool for determining if antimicrobial treatments are working to combat the infection. Since the presence of severe infection instigates two competing isotopic mechanisms that work in opposing directions, the variance in untreated or inappropriately treated individuals is higher than it is in similar individuals without infections. When antimicrobial treatments are applied to infected individuals, the variation in BDV following the treatment returns to a variance similar to the uninfected individuals. A biomarker of the appropriateness of antimicrobial treatment will be of significant value to aid clinicians in antimicrobial stewardship. In the era of competing goals of early intervention in sepsis while limiting antibiotic exposure, a tool to rapidly diagnose and appropriately treat individuals is truly valuable.

The exhaled ¹³CO₂/¹²CO₂ breath delta value has been shown to be a marker for appropriate antimicrobial treatment, a tool that will aid clinicians in determining appropriate treatments, and assist in antimicrobial stewardship.

The use of the terms “a” and “an” and “the” and similar referents (especially in the context of the following claims) are to be construed to cover both the singular and the plural, unless otherwise indicated herein or clearly contradicted by context. The terms first, second etc. as used herein are not meant to denote any particular ordering, but simply for convenience to denote a plurality of, for example, layers. The terms “comprising”, “having”, “including”, and “containing” are to be construed as open-ended terms (i.e., meaning “including, but not limited to”) unless otherwise noted. Recitation of ranges of values are merely intended to serve as a shorthand method of referring individually to each separate value falling within the range, unless otherwise indicated herein, and each separate value is incorporated into the specification as if it were individually recited herein. The endpoints of all ranges are included within the range and independently combinable. All methods described herein can be performed in a suitable order unless otherwise indicated herein or otherwise clearly contradicted by context. The use of any and all examples, or exemplary language (e.g., “such as”), is intended merely to better illustrate the invention and does not pose a limitation on the scope of the invention unless otherwise claimed. No language in the specification should be construed as indicating any non-claimed element as essential to the practice of the invention as used herein.

While the invention has been described with reference to an exemplary embodiment, it will be understood by those skilled in the art that various changes may be made and equivalents may be substituted for elements thereof without departing from the scope of the invention. In addition, many modifications may be made to adapt a particular situation or material to the teachings of the invention without departing from the essential scope thereof. Therefore, it is intended that the invention not be limited to the particular embodiment disclosed as the best mode contemplated for carrying out this invention, but that the invention will include all embodiments falling within the scope of the appended claims. Any combination of the above-described elements in all possible variations thereof is encompassed by the invention unless otherwise indicated herein or otherwise clearly contradicted by context.

1. A method of determining efficacy of an antimicrobial treatment in a subject, comprising
calculating a breath delta value (BDV) for each of at least six breath samples acquired from the subject over a 24 hour period starting from when the subject has been administered the antimicrobial treatment, wherein BDV is determined according to
$$BDV = {δ}^{13} C = \frac{( {}^{13} C / {}^{12} C sample - {}^{13} C / {}^{12} C PDB )}{{}^{13} C / {}^{12} C PDB} \times 1000$$
wherein PDB is a Pee Dee Belemnite reference standard, and BDV is expressed as parts per mil (‰);
calculating a mean standard deviation of BDV (SD BDV) across the six or more breath samples; and
determining that the antimicrobial treatment is effective when the SD BDV is less than or equal to 0.46, or
determining that the antimicrobial treatment is ineffective when the SD BDV is greater than 0.46.

2. The method of claim 1, wherein the subject is an acute surgical or trauma subject admitted to an intensive care unit and suspected of having an infection.

3. The method of claim 2, wherein the subject meets at least two systemic inflammatory response syndrome criteria, infection is suspected based on diagnostic imaging, or infection is suspected based on culture results.

4. The method of claim 1, wherein the subject is intubated.

5. The method of claim 1, wherein it is determined that the antimicrobial treatment is effective and the antimicrobial treatment is continued.

6. The method of claim 1, wherein it is determined that the antimicrobial treatment is ineffective, the antimicrobial treatment is discontinued and a subsequent antimicrobial treatment is initiated; or a subsequent antimicrobial treatment is added to the antimicrobial treatment.

7. The method of claim 1, wherein cavity ringdown spectroscopy is used to measure BDV.

8. The method of v, wherein the antimicrobial is an antibiotic, an antifungal, an antiviral, or an antiparasitic.

9. A method of determining efficacy of an antimicrobial treatment in a subject, comprising
calculating a breath delta value (BDV) for each of at least six breath samples acquired from the subject over a 24 hour period starting before the subject has been administered the antimicrobial treatment, and calculating a breath delta value (BDV) for each of at least six breath samples acquired from the subject over a 24 hour period starting from when the subject has been administered the antimicrobial treatment, wherein BDV is determined according to
$$BDV = {δ}^{13} C = \frac{( {}^{13} C / {}^{12} C sample - {}^{13} C / {}^{12} C PDB )}{{}^{13} C / {}^{12} C PDB} \times 1000$$
wherein PDB is a Pee Dee Belemnite reference standard, and BDV is expressed as parts per mil (‰);
calculating a mean standard deviation of BDV (SD BDV) across the six or more samples acquired before the subject has been administered the antimicrobial treatment, calculating an SD BDV across the six or more samples acquired after the subject has been administered the antimicrobial treatment, and calculating a % decrease in BDV by subtracting the SD BDV after the antimicrobial treatment from the SD BDV before the antimicrobial treatment; and
determining that the antimicrobial treatment is effective when the % decrease in BDV is greater than or equal to 34%, or
Determining that the antimicrobial treatment is ineffective when the % decrease in BDV is less than 34%.

10. The method of claim 9, wherein the subject is an acute surgical or trauma subject admitted to an intensive care unit and suspected of having an infection.

11. The method of claim 10, wherein the subject meets at least two systemic inflammatory response syndrome criteria, infection is suspected based on diagnostic imaging, or infection is suspected based on culture results.

12. The method of claim 10, wherein the subject is intubated.

13. The method of claim 9, wherein it is determined that the antimicrobial treatment is effective and the antimicrobial treatment is continued.

14. The method of claim 9, wherein it is determined that the antimicrobial treatment is ineffective, the antimicrobial treatment is discontinued and a subsequent antimicrobial treatment is initiated; or a subsequent antimicrobial treatment is added to the antimicrobial treatment.

15. The method of claim 9, wherein cavity ringdown spectroscopy is used to measure BDV.

16. The method of claim 9, wherein the antimicrobial is an antibiotic, an antifungal, an antiviral, or an antiparasitic.

17. A method of treating a subject in need of antimicrobial treatment, comprising
administering an initial antimicrobial treatment to the subject;
acquiring at least six breath samples from the subject over a 24 hour period starting from when the subject has been administered the initial antimicrobial treatment;
calculating a breath delta value (BDV) for each of the breath samples according to
$$BDV = {δ}^{13} C = \frac{( {}^{13} C / {}^{12} C sample - {}^{13} C / {}^{12} C PDB )}{{}^{13} C / {}^{12} C PDB} \times 1000$$
wherein PDB is a Pee Dee Belemnite reference standard, and BDV is expressed as parts per mil (‰);
calculating a mean standard deviation of BDV (SD BDV) across the six or more samples; and
determining that the initial antimicrobial treatment is effective when the SD BDV is less than or equal to 0.46, and continuing administering the initial antimicrobial treatment, or
determining that the antimicrobial treatment is ineffective when the SD BDV is greater than 0.46, discontinuing the initial antimicrobial treatment, and administering a subsequent antimicrobial treatment, or adding a subsequent antimicrobial treatment to the antimicrobial treatment.

18. The method of claim 17, wherein the subsequent antimicrobial treatment targets a different class of pathogen than the initial antimicrobial treatment.

19. The method of claim 17, wherein the initial and subsequent antimicrobial treatment are an antibiotic, an antifungal, an antiviral, or an antiparasitic.

20. The method of method of claim 17, wherein the subject is an acute surgical or trauma subject admitted to an intensive care unit and suspected of having an infection.

21. The method of claim 20, wherein the subject meets at least two systemic inflammatory response syndrome criteria, infection is suspected based on diagnostic imaging, or infection is suspected based on culture results.

22. The method of method of claim 17, wherein the subject is intubated.

23. The method of method of claim 19, wherein cavity ringdown spectroscopy is used to measure BDV.

24. The method of method of claim 19, wherein, prior to administering an initial antimicrobial treatment to the subject, the method comprises calculating a breath delta value (BDV) for at least two pre-infection samples, and determining that the subject has an infection when a change in BDV of greater than or equal to 1.0‰ is determined.

25. A method of treating a subject in need of antimicrobial treatment, comprising
acquiring at least six breath samples from the subject over a 24 hour period;
administering an initial antimicrobial treatment to the subject;
acquiring at least six breath samples from the subject over a 24 hour period starting from when the subject has been administered the initial antimicrobial treatment;
calculating a breath delta value (BDV) for each of the breath samples acquired before and after the initial antimicrobial treatment according to
$$BDV = {δ}^{13} C = \frac{( {}^{13} C / {}^{12} C sample - {}^{13} C / {}^{12} C PDB )}{{}^{13} C / {}^{12} C PDB} \times 1000$$
wherein PDB is a Pee Dee Belemnite reference standard, and BDV is expressed as parts per mil (‰);
calculating a mean standard deviation of BDV (SD BDV) across the six or more samples acquired before the subject has been administered the initial antimicrobial treatment, calculating an SD BDV across the six or more samples acquired after the subject has been administered the initial antimicrobial treatment, and calculating a % decrease in BDV by subtracting the SD BDV after the initial antimicrobial treatment from the SD BDV before the initial antimicrobial treatment; and
determining that the antimicrobial treatment is effective when the % decrease in BDV is greater than or equal to 34%, and continuing administering the initial antimicrobial treatment, or
determining that the antimicrobial treatment is ineffective when the % decrease in BDV is less than 34%, discontinuing the initial antimicrobial treatment and administering a subsequent antimicrobial treatment, or adding a subsequent antimicrobial treatment to the antimicrobial treatment.

26. The method of claim 24, wherein the subsequent antimicrobial treatment targets a different class of pathogen than the initial antimicrobial treatment.

27. The method of method of claim 24, wherein the initial and subsequent antimicrobial treatment are an antibiotic, an antifungal, an antiviral, or an antiparasitic.

28. The method of method of claim 24, wherein the subject is an acute surgical or trauma subject admitted to an intensive care unit and suspected of having an infection.

29. The method of claim 27, wherein the subject meets at least two systemic inflammatory response syndrome criteria, infection is suspected based on diagnostic imaging, or infection is suspected based on culture results.

30. The method of method of claim 27, wherein the subject is intubated.

31. The method of method of claim 24, wherein cavity ringdown spectroscopy is used to measure BDV.
